(19) United States
(12) Patent Application Publication
(10) Pub. No.: US 2023/0183756 A1
(43) Pub. Date: Jun. 15, 2023

(54) PROCESS FOR THE BIOREACTIVE EXTRACTION OF PRODCUCED OXYGEN FROM A REACTION CHAMBER, AND USE OF PHOTOTROPHIC MICRO-ORGANISMS IN THE RECOVERY OF HYDROGEN

(71) Applicant: HELMHOLTZ-ZENTRUM FUER UMWELTFORSCHUNG GMBH - UFZ, Leipzig (DE)

(72) Inventors: Andreas Schmid, Leipzig (DE); Anna Hoschek, Leipzig (DE); Bruno Buehler, Leipzig (DE)

(21) Appl. No.: 16/490,170

(22) PCT Filed: Mar. 6, 2018
(86) PCT No.: PCT/EP2018/055449

§ 371 Date: Oct. 25, 2019

(30) Foreign Application Priority Data
Mar. 6, 2017 (DE) 10 2017 104 648.8

(51) Int. Cl. C12P 7/04; C12P 3/00; C12M 1/00; C12N 9/02

(52) U.S. Cl. CPC C12P 7/04; C12P 3/00; C12M 21/02; C12M 47/18; C12N 9/0073; C12Y 114/13069

(57) ABSTRACT

The invention relates to a method for extracting oxygen form a reaction chamber, as well as a use of microorganisms in such a method and a photobioreactor for carrying out the method.

According to the invention, the method comprises the steps—irradiating at least one phototropic microorganism with light under anaerobic conditions in a reaction chamber, and—in situ bonding of produced oxygen by means of the microorganism by oxidation of an oxygen-utilizing substrates.

The invention relates to a method for the bioreactive extraction of produced oxygen from a reaction chamber.

### PRIOR ART

Hydrogen is the energy carrier of the future, as on the one hand it has a high energy density and on the other hand represents an environmentally friendly energy source. It is available in limitless quantities and can be obtained from water using various methods. Hydrogen can be converted back into current and heat in a fuel cell, with the end product in turn being just water. At least theoretically, enormous quantities of energy can also be buffered in the long term by means of hydrogen.

In particular, electrolysis and photocatalytic water splitting are to be named for producing hydrogen.

Electrolysis is a well-known and proven method for producing hydrogen. Water is broken down into hydrogen and oxygen using electrical current. At present, this method is economical only if there is sufficiently cost-effective current available.

Photocatalytic water splitting-splitting water into hydrogen and oxygen by means of sunlight-represents a technologically important alternative for energy-expensive electrolytic water splitting. In this case, generally, algae or cyanobacteria are used which, under certain conditions, can give off hydrogen to the environment during the metabolic process. In this way, sunlight and water can be converted directly into hydrogen by means of photosynthesis in algae reactors. The enzymatic process consists of two steps. In the first step, the water is divided by the photosynthetic light reaction into protons, electrons and molecular oxygen. In the second step, the protons are reduced to molecular hydrogen by, for example, hydrogenases. In this oxygenic, i.e. oxygen-releasing, photosynthesis, two large, cofactor-containing protein complexes, photosystem I (PSI) and photosystem II (PSII) work together. With the help of light energy, PSII splits water into protons, oxygen and electrons, and transfers the latter to PSI via an electron transport chain.

Coupling the hydrogenase with the photosystem I, as well as the water-splitting photosystem II of the microorganisms, subsequently makes possible production of hydrogen from light and water in which only oxygen accrues as “by-product”. When oxygen and hydrogen converge, there is a risk of explosion due to the formation of oxyhydrogen gas. Additionally, known hydrogenases are oxygen-sensitive. Therefore, it is first necessary to separate the gases quickly after formation in order to reduce the risk of explosion (production of oxyhydrogen gas) and the degeneration of the hydrogenases. This takes place for example using oxygen-resistant hydrogenases (US 20090263846 A1), porous membranes (DE 102007002009 A1) or temporary separation of oxygen formation and hydrogen production (U.S. Pat. No. 4,532,210 A).

These techniques possess substantial disadvantages, as the oxygen-resistant hydrogenases do not suppress the formation of oxyhydrogen gas, the membrane technology does not prevent the inactivation of an oxygen-sensitive enzyme system and, by temporarily separating oxygen formation and hydrogen production, the method is separated into a productive and non-productive phase. Accordingly, photocatalytic hydrogen production is associated, on a commercial scale, with specific conditions which cannot be achieved in terms of production technology, or can be achieved only at high cost.

Therefore, the object of the invention is to provide a method for producing hydrogen which separates undesirably produced molecular oxygen from the chemical production of a target product and prevents the disadvantages of the prior art, such as the risk of explosion due to the formation of oxyhydrogen gas, two process phases and inactivation of oxygen-sensitive enzymes. In particular, the object is to find environmentally-friendly, inexpensive and stable photocatalysts which can bind photosynthetically produced oxygen during hydrogen production.

The object is achieved by a method for the bioreactive extraction of photocatalytically produced oxygen in a reaction chamber, comprising the following steps:
irradiating at least one phototrophic microorganism with light under anaerobic conditions in a reaction chamber, and
in situ bonding of produced oxygen by the microorganism.

The method according to the invention overcomes technical requirements which result from oxygen-sensitive processes or educts, products and enzymes. Oxygen-stable microorganisms and enzymes can thus be used also in oxygen-producing enzymatic methods without performance being impaired. Processes in which both hydrogen and oxygen form, and which thus potentially produce oxyhydrogen gas, are deactivated as both reaction partners are separated in-situ.

This is possible due to the oxygen being captured and enzymatically bonded directly where it is produced. The resulting oxygen is thus deactivated directly where it is produced. The deactivation takes place for example due to the oxidation of a substrate likewise present in the reaction chamber in the form of a chemical bonding of the oxygen to the substrate, because of which the oxygen is no longer displaced back into the reaction chamber.

Therefore, the advantage consists in particular of the oxygen being not only separated or masked but chemically deactivated instead. The reaction chamber and in particular the microorganism thus preferably also have an oxygen-utilizing substrate.

Furthermore, the invention makes it possible to regulate oxygen deactivation quantitatively by controlling the oxygen-consuming reaction. A previously defined quantity of oxygen, which lies in the range of 0 (produced oxygen completely bonded) to 1 (no oxygen bonded), can thus be made available to the reaction chamber.

By making a suitable choice of substrate, an oxidized target product can also be produced, with the result that the bonding of the disruptive oxygen produced in the actual process will lead to a further step of adding value.

The resulting oxygen is, at the moment, molecular oxygen.

The microorganism preferably has or produces an oxygen-converting enzyme.

In a further preferred embodiment, the preferred target product is hydrogen or protons which are then reduced with electrons to hydrogen molecules. Alternatively, the target product is methanol which is produced by reducing CO₂ with the electrons which come from water splitting. Both cases concern a fuel which is used for example as a starting product for a fuel cell reaction. These reactions are for example catalyzed by three dehydrogenases which belong to the class of oxidoreductases.

CO₂+2e⁻→formiate (I)

Formiate+2e⁻→formaldehyde (II)

Formaldehyde+2e⁻→methanol (III)

In particular, the at least one phototropic microorganism therefore has an enzyme selected from the group hydrogenase, nitrogenase and/or oxidoreductase.

The method is preferably carried out in a liquid, preferably aqueous, medium. In a preferred embodiment it is provided to remove disrupting oxygen (O₂), which is produced during the photosynthetic light reaction from water or aqueous liquids or solutions using phototropic microorganisms in the reaction chamber, from the reaction chamber in a process. In so doing, the phototropic microorganism has or produces at least one oxygen-converting enzyme. At the same time, oxygen-utilizing substrates are introduced.

Advantageously, in addition to the hydrogenase, nitrogenase and/or oxidoreductase, the microorganism has at least one further, oxygen-converting enzyme. In particular in a combination of hydrogenase and an oxidoreductase, the hydrogen-producing and the oxygen-converting process step takes place in the same microorganism, and thus without spatial separation. The oxygen is converted directly after its production, and deactivated according to the invention. In a preferred embodiment it is provided that the further enzyme is an oxidoreductase, in particular oxidase and oxygenase.

Alternatively, in the reaction chamber there is a further microorganism which has the further enzyme. In other words, preferably one microorganism has hydrogenase, nitrogenase and/or oxidoreductase as hydrogen-producing enzyme and additionally oxidoreductase as oxygen-converting enzyme, or two different microorganisms present in the reaction chamber each have one of these enzymes. Here, only a combination of the named enzymes is mentioned explicitly, wherein all other combinations are also expressly provided. In the embodiment of different microorganisms, the oxygen-producing and the oxygen-converting process take place not in the same microorganism, but nevertheless in the same reaction chamber. The advantage is in particular that the oxygen-converting process, and thus the quantitative amount of oxygen present in the process, can be controlled by a quantitative ratio of microorganisms to one another.

Furthermore, it is preferred that the microorganism itself produces the target product, thus for example hydrogen or methanol. In other words, in a preferred embodiment of the method according to the invention, in addition to oxygen, hydrogen or methanol is also released during irradiation of the at least one phototropic microorganism.

The method according to the invention thus provides, in a preferred embodiment, that at least one phototropic microorganism is brought into contact with water or an aqueous liquid or solution, accompanied by exposure to light, wherein a microorganism is selected which, in addition to the hydrogenases, also has or produces oxidoreductases as oxygen-converting enzymes, or a further microorganism in the same reaction chamber is selected which has or produces oxidoreductases as oxygen-converting enzymes.

The oxidoreductase is preferably selected from the group of oxidases or oxygenases, preferably monooxygenases, dioxygenases or coenzyme-independent oxygenases. An enzyme consuming monooxygenase as oxygen should be the enzyme of choice if the stoichiometric equilibrium in the reaction chamber is of interest. This results in the following reaction system:

The photosynthetic light reaction:2H₂O→4H⁺+O₂+4e⁻ (IV)

Reaction of the monooxygenase:S+O₂+2e⁻+2H⁺→_{oxidized}S+H₂O (V)

Reaction of the hydrogenase:2H⁺2e⁻→H₂ (VI)

Oxidoreductases of the type AlkB, which are known in the literature as catalysts for producing predominantly less markedly oxidized products, are particularly preferred. These are particularly suitable for producing predominantly products of higher oxidation stages from alkane and the carboxylic acids or carboxylic acid esters thereof. Such oxidoreductases are also qualified for selective oxidation of alkanes and the carboxylic acid or carboxylic acid esters thereof, wherein by-products to be expected, in particular at other alkanes oxidized as terminal carbon atoms, are produced only on an unexpectedly small scale or in entirely undetectable quantities.

Here, the substrates are oxidized preferably using an AlkB-type oxidoreductase in the presence of oxygen. AlkB represents an oxidoreductase which became known firstly from the AlkBGT system from Pseudomonas putida Gpo1, which oxidoreductase is dependent on two further polypeptides, AlkG and AlkT. AlkT is characterized as FAD-dependent rubredoxin reductase which relays the electrons from NADH to AlkG. AlkG is a rubredoxin, a ferruginous redox protein which functions as a direct electron donor for AlkB. In a preferred embodiment, the same term “AlkB-type oxidoreductase” means a polypeptide with a sequence homology of increasingly preferably at least 75, 80, 85, 90, 92, 94, 96, 98 or 99% to the sequence of the AlkB of Pseudomonas putida Gpo1 (database code: CAB54050.1; this database code comes from the prior art, specifically from the NCBI database, more precisely from the release available online on 15 Nov. 2011) with the ability to oxidize the substrates according to the invention, such as alkanes and the carboxylic acids thereof. In a particularly preferred embodiment, the AlkB-type oxidoreductase is an alkane-oxidizing oxidoreductase which functionally interacts with the AlkG (CAB54052.1) and AlkT (CAB54063.1) polypeptides of Pseudomonas putida Gpo1. In a most preferred embodiment, the AlkB-type oxidoreductase is AlkB from the AlkBGT system from Pseudomonas putida Gpo1 or a variant thereof.

Particularly advantageously, substrates are used which continue to be used after the oxidation as educts and form so-called value added products for the method according to the invention or are those which are undesirable in the reaction chamber and are deactivated by the oxidation. Thus, in addition to the production of the target product, such as hydrogen or methanol, oxygen conversion is also a target process of the method. Then it no longer concerns only the bonding of undesired oxygen which disrupts production of the first target product, but the bonding of resulting oxygen to a second target product. An example of use in which an undesired educt is deactivated by the method according to the invention is wastewater treatment. Here, different substrates are present as undesired substances in the wastewater. The wastewater is purified by oxidizing these substrates. Depending on the microorganism used, hydrogen as fuel, for example, is produced as desired by-product.

In a preferred embodiment, carbon compounds which are added or found in the aqueous starting solution function as substrates in the method according to the invention. These are preferably carboxylic acids or carboxylic acid esters of medium-chained alkanes with a C₃-C₂₀, in particular C₅-C₁₅, preferably C₇-C₁₀ chain. A particularly preferred example is methyl nonanoate (C₈H₁₇—COOH—CH₃).

The method according to the invention preferably provides that algae, purple bacteria or cyanobacteria are used as phototropic microorganism, wherein cyanobacteria are preferred. These perform photosynthesis and have oxygen-converting enzymes, produce these or are suitable, in appropriate levels, for genetic modification, in order to have or produce such enzymes.

Particularly advantageously a, in particular genetically modified, cyanobacterial strain is used which has the alkane monooxygenase enzyme system AlkBGT.

In a particularly preferred embodiment of the method according to the invention, the genetically modified cyanobacterial strain Synechocystis sp. PCC6803 is used as microorganism. This has the alkane monooxygenase enzyme system AlkBGT.

A further aspect of the invention thus relates to a cyanobacteria cell comprising a hydrogenase and the alkane monooxygenase of alkBGT coded with SEQ ID NO: 1 (from Pseudomonas putida GPo1) or a variant thereof, in particular an enzyme which is at least 80% identical to SEQ ID NO: 1.

The teaching of the present invention cannot be carried out only using the exact amino acid or nucleic acid sequences of the biological macromolecules described herein, but also using variants of such macromolecules which can be obtained by deletion, addition or substitution of one or more as an amino acid or nucleic acid.

in a preferred embodiment, the term “variant” of a nucleic acid sequence or amino acid sequence used below synonymously and interchangeably with the term “homologs”, as used herein, means another nucleic acid or amino acid sequence which, with respect to the corresponding original wild-type nucleic acid or amino acid sequence, has a homology, here used synonymously with identity, of 70, 75, 80, 85, 90, 92, 94, 96, 98, 99% or more percent, wherein preferably amino acids other than those forming the catalytically active center or essential to the structure or folding are deleted or substituted or the latter are merely conservatively substituted, for example a glutamate instead of an aspartate or a leucine instead of a valine. There is no need for the sequence to have a corresponding high homology over its entire length since, according to the invention, fusion proteins or nucleic acids coding for such can be used which have a part with corresponding homology and/or activity. The prior art describes algorithms which can be used to calculate the extent of homology of two sequences, e.g. Arthur Lesk (2008), Introduction to bioinformatics, 3rd edition.

In a further preferred embodiment of the present invention, the variant of an amino acid or nucleic acid sequence has, preferably in addition to the above-named sequence homology, substantially the same enzymatic activity of the wild-type molecule or the original molecule. For example, a variant of a polypeptide enzymatically active as protease has the same or substantially the same proteolytic activity as the polypeptide enzyme, i.e. the capacity to catalyze the hydrolysis of a peptide bond. In a particular embodiment, the term “substantially the same enzymatic activity” means an activity with respect to the substrates of the wild-type polypeptide which is clearly above the background activity or/and differs by less than 3, more preferably 2, even more preferably an order of magnitude of KM and/or kcat values which the wild-type polypeptide possesses with respect to the same substrates. In a further preferred embodiment, the term “variant” of a nucleic acid or amino acid sequence comprises at least one active part/or fragment of the nucleic acid or amino acid sequence.

In particular in the above-named embodiment methyl nonanoate is used as substrate.

A further aspect of the invention relates to the use of phototropic microorganisms which, in addition to hydrogenases, have or produce oxidoreductases as oxygen-converting enzymes when obtaining hydrogen (H₂) and oxygen (O₂) from water or aqueous liquids and solutions, and for simultaneous, in-situ removal of the oxygen (O₂) using corresponding substrates which form products with the oxygen. Preferably, the invention relates to the use of phototropic microorganisms which, in addition to hydrogenases, have or produce oxidoreductases as oxygen-converting enzymes in the method according to the invention.

The use of the method according to the invention is particularly preferred in wastewater treatment. In so doing, a mixture of undefined organic substances, which act as substrates within the scope of the invention, is made available for the oxygen-utilizing reaction of the microorganism. The choice of a non-specific, non-selective oxygenation reaction makes possible the oxidation and thus the inactivation of the contaminants occurring in the wastewater. In so doing, a production of hydrogen gas, advantageously simultaneously as source for energy generation, and an oxidation of dissolved organic carbon, takes place.

Preferably, an algae strain, a purple bacterial strain or a cyanobacterial strain is used as microorganism. Particularly preferably, a strain which has the alkane monooxygenase enzyme system AlkBGT, such as for example Synechocystis sp. PCC6803, is used.

The method according to the invention is preferably carried out in the photobioreactor. A further aspect of the invention thus relates to a photobioreactor for carrying out the method according to the invention, in particular comprising the genetically modified cyanobacterial strain Synechocystis sp. PCC6803 which has or produces the alkane monooxygenase enzyme system AlkBGT. In this case, the microorganism, in particular the bacterial strain, is present as biofilm.

Photobioreactors are fermenters in which the phototropic microorganisms, for example algae, cyanobacteria and purple bacteria, are cultivated, in which thus either the growth and the multiplication of these cells is made possible, or the production of different substances by means of phototropic cells is promoted. Unlike conventional biotechnical fermentation processes, phototropic processes are light-dependent. In order for the cells to make an active metabolism possible, they are optimally supplied both with light and with different nutrient solutions or dissolved substrates.

The photobioreactors vary considerably in material and construction method. Common to all of them is that, on the basis of their structure, they are intended to guarantee optimum light supply of the microorganisms. For example, reactors which work with plastic bags or tubes or systems which, with flat containers or tubes, expose biomass to the sun in thin layers, are available on the market.

Each reactor also has, in addition to the light-permeable photosynthesis part, a feed for CO₂ and substrates and a device for harvesting the microorganisms. Mostly, the state and density of the culture is still detected via a sensitive measuring electronics in order to be able to intervene in a regulatory manner.

The tubular reactor is a matured photobioreactor already in use in large-scale plants. The microorganism suspension is conveyed in transparent tubes made of glass or plastic, and held in motion by means of a pump.

The helix or coil reactor is basically a tubular reactor which however does not use any glass tubes but a flexible tube instead. This is then arranged in a circular manner, and the process control is similar to the tubular reactor.

The bag or tube reactor is a photobioreactor based on individual compartments of plastic film, often arranged suspended. Approaches using functional plastics and ideas to position the bag on the water can also be used here. Thorough mixing takes place mostly due to the CO₂ input, meaning it is thus relatively energy-efficient. The film is not directed to a long-term use, but is instead reconditioned from time to time.

In the boiler reactor, a conventional column-shaped liquid container is used in which the algae biomass is bred. Depending on the design of the container, light is introduced into the container via different systems. This can be sunlight or artificial light sources. Some approaches use lens systems and optical conductors for this. A thorough mixing can take place via different mechanisms, such as for example gas input or agitation by pumps.

In the flat plate reactor, the microorganisms are exposed to light in flat layers in the culture medium. The reactor is bounded by glass or plastic plates, while thorough mixing takes place due to the gas input.

In the thin layer reactor, the principle of minimizing the microorganism layer for optimal luminous efficacy continues to be pursued consistently. This is intended to assist in minimizing the self-overshadowing of denser suspensions. In so doing, immobilized microorganisms are exposed to light on a carrier material.

According to the invention, tubular reactors are preferably used.

In summary, the invention makes possible a combination of two techniques which have thus far been independent: photocatalytic hydrogen gas production and photocatalytic oxygen reaction. At the same time as producing hydrogen as biofuel, a substrate is oxidized and deactivated or a value added product is produced.

The present invention is further illustrated by the following Figures and non-limiting examples, from which further features, embodiments, aspects and advantages of the present invention can be seen.

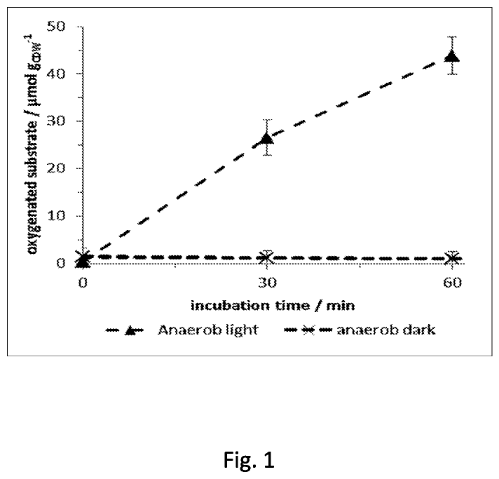

FIG. 1: graphic representation of an extraction of photosynthetically produced oxygen by the alkane monooxygenase enzyme system AlkBGT which has been introduced genetically into the cyanobacterial strain Synechocystis sp. PCC6803, in a manner carried out according to the invention.

Different conditions come into question for carrying out the method according to the invention. The only essential is the presence of molecular oxygen as oxidizing agent.

### Example 1

Production of a genetically modified strain—Synechocystis sp. PCC6803, containing the alkane monooxygenase enzyme system AlkBGT

The introduction of the gene coding for the alkane monooxygenase enzyme system AlkBGT took place via a plasmid-based approach (pRSF_Ptrc1O:BGTII). The following protocols and cloning steps describe the structure of the plasmid. Table 1 lists the strains and plasmids used and produced during the cloning process.

TABLE 1
Strain/plasmid structures used and produced during the cloning process.

| Strain/plasmid | Description | Reference |
|---|---|---|
| E. coli DH5α | F⁻ Φ80lacZΔM15 Δ(lacZYA-argF) U169 | (Hanahan |
| | recA1 endA1 hsdR17 (rK⁻, mK⁺) phoA | 1983) |
| | supE44 λB⁻ thi⁻¹ gyrA96 relA1 | |
| Synechocystis sp. PCC6803 | Geographical origin: California, USA | (Stanier |
| | Obtained from the Pasteur Culture | et al. 1971) |
| | Collection of Cyanobacteria (PCC, Paris, | |
| | France) | |
| pBT10 | Alkane monooxygenase expression | (Schrewe |
| | system (alkBFG, alkST) in pCOM10 | et al. 2011) |
| pSB1AC3_Ptrc1O:GFPmut3B | P_{trc1O} promoter, GFPmut3B gene | (Huang et |
| | (BBa_E0040) in pSB1AC3 | al. 2010) |
| pSB1AC3_Ptrc1O:Term | pSB1AC3 with the p_{trc1O} promoter from | This paper |
| | pSB1AC3_Ptrc1O:GFPmut3B via XbaI, | |
| | PstI (Gibson assembly) | |
| pSB1AC3_PrnpB:lacI | PrnpB (constitutive promoter of the | (Huang et |
| | RNase P gene) controlling a variant of | al. 2010) |
| | the lac repressor lacI in pSB1AC3 | |
| pSB1AC3_PrnpB:lacI_Ptrc1O:Term | pSB1AC3_Ptrc1O:Term with PrnpB:lacI | This paper |
| | from pSB1AC3_PrnpB:lacI via XbaI | |
| | (Restriction, Ligation) | |
| pPMQAK1 | Broad host range Plasmid, RSF ori, mob | (Huang et |
| | gene | al. 2010) |
| pPMQAK1_PrnpB:lacI_Ptrc1O:Term | pPMQAK1 with PrnpB:lacI_Ptrc1O:Term | This paper |
| | from pSB1AC3_PrnpB:lacI_Ptrc1O:Term | |
| | via EcoRI, PstI (Restriction, Ligation) | |
| pPMQAK1_PrnpB:lacI_Ptrc1O:BGTII | pPMQAK1_PrnpB:lacI_Ptrc1O:Term with | This paper |
| | alkBGT genes from pBT10 (successive | |
| | genes, optimized RBS, C-terminal Strep- | |
| | tag II) via Spel (Gibson assembly) | |
| pRSF_Ptrc1O:BGTII | pPMQAK1_PrnpB:lacI_Ptrc1O:BGTII | This paper |
| | with additional terminator (biobrick | |
| | #BBa_B0015 via XbaI (Gibson | |
| | assembly) | |

As part of the cloning process, the following method steps such as restriction, amplification etc. were carried out as below.

The restriction endonucleases were obtained from Thermo Scientific—Germany GmbH (Schwerte, Germany) and used as recommended.

An amplification of DNA fragments was carried out by polymerase chain reaction (PCR) applying the Phusion High Fidelity (HF) DNA polymerase from Thermo Scientific—Germany GmbH (Schwerte, Germany) via the recommended 3-step protocol with corresponding primers (listed in Table 2). Corresponding accumulation temperatures (T_{An}) and elongation times (t_{EL}) are described below in the respective process step.

A desired overlap extension PCR (OEPCR) was carded out by inserting 50 ng each of DNA fragments in a 100 μL standard PCR batch. Corresponding primers were added after 5 PCR cycles.

Plasmid DNA was dephosphorylated by means of FastAP Thermosensitive Alkaline Phosphatase from Thermo Scientific— Germany GmbH (Schwerte, Germany) as recommended.

Subsequently, plasmid DNA and amplified DNA fragments were purified via the PCR Clean-up Gel Extraction Kit from MACHERY-NAGEL GmbH & Co. KG (MACHEREY-NAGEL GmbH & Co. KG, Düren, Germany).

Gibson assembly took place via a single-step isothermal, in vitro recombinant cloning method described by Gibson et al. 2009 (Gibson et al. 2009).

Ligation took place via the T4 DNA ligase from Thermo Scientific—Germany GmbH (Schwerte, Germany) as recommended.

Finally, PCR-amplified DNA sequences were verified by sequencing via Eurofins MWG (Ebersberg, Germany).

The present paper refers to the known processes named in Table 1 with respect to the reference noted in Table 1.

TABLE 2
Primers used during the cloning process.

| | SEQ.- | |
|---|---|---|
| Primer# | No. | sequence |
| PAH055 | 1 | TCCGGCTCGTATAATGTGTG |
| | | GAATTGTGAGCGGATAACAA |
| | | TTTCACACATACTAGTACCA |
| | | GGCATCAAATAAAACG |
| PAH056 | 2 | TATAAACGCAGAAAGGCCC |
| PAH057 | 3 | TGATTTCTGGAATTCGCGGC |
| | | CGCTTTCTAGATTGACAATT |
| | | AATCATCCGGCTCGTATAAT |
| | | GTG |
| PAH058 | 4 | ACACCTTGCCCGTTTTTTTG |
| | | CCGGACTGCAGTATAAACGC |
| | | AGAAAGGCCC |
| PAH069 | 5 | CTTTTCCTCGTAGAGCAC |
| PAH070 | 6 | GAGCCACCCGCAGTTCGAAA |
| | | AATAGTACTAGAGTAGTGGA |
| | | GGTTACTAGATGGCAATCGT |
| | | TGTTGTTG |
| PAH071 | 7 | ATCAGGTAATTTTATACTCC |
| | | C |
| PAH072 | 8 | CTATTTTTCGAACTGCGGGT |
| | | GGCTCCAAGCGCTCTTTTCC |
| | | TCGTAGAGCAC |
| PAH073 | 9 | CTTTCGTTTTATTTGATGCC |
| | | TGGTACTATTTTTCGAACTG |
| | | CGGGTGGCTCCAAGCGCTAT |
| | | CAGGTAATTTTATACTCCC |
| PAH077 | 10 | GGGAGGTATTGGACCGCATT |
| | | GAACTCTAGTATATAAACGC |
| | | AGAAAGGCCC |
| PAH078 | 11 | ACGAGCCGGATGATTAATTG |
| | | TCAATCTAGAGCCAGGCATC |
| | | AAATAAAACG |
| SPAH017 | 12 | CCATCAAACAGGATTTTCG |
| SPAH023 | 13 | TGCCACCTGACGTCTAAGA |
| | | A |

Cloning Process

Structure of pSB1AC3 Ptrc1O:Term

Restriction: pSB1AC3_Ptrc1O:GFP (Xbal+Pstl)→pSB1AC3 (Xbal, Pstl)

Amplification: Ptrc1O:Term part I of pSB1AC3_Ptrc1O:GFP
(PAH055+PAH056→186 BP, T_{An}: 60° C., t_{Elong}: 10 sec)
Ptrc1O:Term part II of part I
(PAH057+PAH058→262 BP, T_{An}: 72° C., t_{Elong}: 10 sec)

Gibson assembly: pSB1AC3 (Xbal, Pstl)+Ptrc1O:Term part II
→pSB1AC3 Ptrc1O:Term

Structure of pSB1AC3 PrnpB:lacl Ptrc1O:Term

Restriction: pSB1AC3_Ptrc1O:Term (Xbal)

Dephosphorylation: pSB1AC3_Ptrc1O:Term (Xbal) (FastAP, Thermo)

Restriction: pSB1AC_PrnpB:lacl (Xbal+Spel)

Ligation: pSB1AC3_Ptrc1O:Term (Xbal)+PrnpB:lacl (Xbal_Spel) (1:2)
→pSB1AC3 PrnpB:lacl Ptrc1O:Term

Verification by PCR: Clones with the PrnpB:lacl fragment in desired direction determined by PCR (SPAH017+SPAH023→500 BP, T_{An}: 61° C., t_{Elong}: 15 sec).

Structure of pPMQAK1 PrnpB:lacl Ptrc1O:Term

Restriction: pPMQAK1 (EcoRI+Pstl)

Restriction: pSB1AC3_PrnpB:lacl_Ptrc1O:Term (EcoRI+Pstl)

Ligation: pPMQAK1 (EcoRI, PstI)+PrnpB:lacl_Ptrc1O:Term (EcoRI, PstI) (1:5)
→pPMQAK1 PrnpB:lacl Ptrc1O:Term

Structure of pPMQAK1_PrnpB:lacl Ptrc1O:BGTII

Restriction: pPMQAK1_PrnpB:lacl_Ptrc1O:Term (Spel)

Amplification: oAlkBII (PAH059+PAH067→1283 BP, T_{An}: 65° C., t_{Elong}: 25 sec)
oAlkGII (PAH068+PAH069→568 BP, T_{An}: 60° C., t_{Elong}: 25 sec)
oAlkTII (PAH070+PAH071→1204 BP, T_{An}: 65° C., t_{Elong}: 25 sec)

OE-PCR: oAlkBII+oAlkGII (PAH063+PAH072→1859 BP, T_{An}: 65° C., t_{Elong}: 60 sec)
→oBGII
oBGII+oAIkTII (PAH063+PAH073→3096 BP, T_{An}: 57° C., t_{Elong}: 90 sec)
→oBGTII

Gibson: pPMQAK1_PrnpB:lacl_Ptrc1O:Term (Spel)+oBGT
→pPMQAK1 PrnpB:lacl Ptrc1O:BGTII

Structure of pRSF Ptrc1O:BGTII

Restriction: pPMQAK1_PrnpB:lacl_Ptrc1O:BGTII (Xbal)

Amplification: Term of pSB1AC3_Ptrc1O:GFP
(PAH077+PAH078→191 BP, T_{An}: 60° C., t_{Elong}: 5 sec)

Gibson: pPMQAK1_PrnpB:lacl_Ptrc1O:oBGTII (Xbal)+Term →pRSF Ptrc1O:BGTII

Growth Conditions for Synechocystis sp. PCC6803

Synechocystis sp. PCC6803 was cultivated in YBG11 medium, based on Shcolnick et al. 2007, upon addition of 50 mM HEPES buffer (Shcolnick et al. 2007). 50 μg mL⁻¹ kanamycin was added as evolutionary pressure. Standard cultivation conditions comprise a culture volume of 20 mL YBG11 medium in 100 mL Erienmeyer shaking flasks with chicane, which was introduced into an orbital shaker (Multitron Pro shaker, Infors, Bottmingen, Switzerland) at 150 rpm (2.5 cm amplitude). The cultivation temperature was 30° C., at a luminous intensity of 50 μmol m⁻² s⁻¹ (LED), 0.04% CO₂ and an air humidity of 75%. Growth was pursued via the optical density at a wavelength of 750 nm over a spectrophotometer (Libra S11, Biochrom Ltd, Cambridge, UK). Preparatory cultures were incubated over 200 μL of a cryostock solution and cultivated under standard conditions for 4-6 days. Main cultures, proceeding from this preparatory culture, were inoculated with a start OD₇₅₀ of 0.08 and cultivated for 3 days under standard conditions until the gene expression was induced for a further day by adding 2 mM IPTG.

YBG11: 1.49 g L⁻¹ NaNO₃, 0.074 g L⁻¹ MgSO₄·7 H₂O, 0.305 g L⁻¹ K₂HPO₄, 10 mL L⁻¹ YBG11 trace elements (100×), 0.019 g L⁻¹ Na₂CO₃, 50 mM HEPES (pH 7.2); YBG11 trace elements (100×): 0.36 g L⁻¹ CaCl₂·2 H₂O, 0.63 g L⁻¹ citric acid, 0.28 g L⁻¹ boric acid, 0.11 g L⁻¹ MnCl₂·4 H₂O, 0.02 g L⁻¹ ZnSO₄·7 H₂O, 0.039 g L⁻¹ Na₂MoO₄·2 H₂O, 0.007 g L⁻¹ CuSO₄·5 H₂O, 0.003 g L-1 Co(NO₃)₂·6 H₂O, 0.1 g L⁻¹ FeCl₃·6 H₂O, 0.6 g L⁻¹ Na₂EDTA·2 H₂O

Transformation of Synechocystis sp. PCC6803 by Electroporation

Transformation of Synechocystis sp. PCC6803 with the plasmid pRSF_Ptrc1O:BGTII was carried out via electroporation, based on a method according to Ferreira et al. 2014 (Universidade do Porto Ferreira 2014). Electrocompetent cells were produced proceeding from a 50 mL YBG11 main culture (in 100 mL Erienmeyer shaking flasks with chicane) with an OD₇₅₀ of 0.5-1. The cells were harvested by centrifugation (10 min, 3180 g, 4° C.), washed three times each with 10 mL HEPES buffer (1 mM, pH 7.5) and resuspended in 1 mL HEPES buffer. The electrocompetent cells were stored by adding 5% (v/v) DMSO at −80° C. For electroporation, 0.2-1.0 μg plasmid DNA was added to 60 μL electrocompetent cells in an electroporation vessel (2 mm electrode distance), pulsed for 5 ms at 2500 V (12.5 kV cm⁻¹) (Eppendorf Eporator, Eppendorf Vertrieb Deutschland GmbH, Wesseling-Berzdorf, Germany) and then transferred into 50 mL YBG11 medium (in 100 mL Erlenmeyer shaking flasks with chicane). After cultivation under standard conditions for 24 h, the cells were harvested by centrifugation (10 min, 3180 g, RT), resuspended in 100 μL YBG11 medium and exposed to BG11 agar plates with 50 μg mL⁻¹ kanamycin (Stanier et al. 1971). After 4-6 days at 30° C., 20-50 μmol m⁻² s⁻¹ luminous intensity (fluorescent tubes), 0.04% CO₂ and 80% air humidity (poly klima GmbH, Freising, Germany), individual colonies were transferred to fresh BG11 agar plates and incubated again. The cell mass of an agar plate was then used for inoculation of a 20 mL YBG11 preparatory culture.

BG11 agar plates: 1.5 g L⁻¹ NaNO₃, 0.075 g L⁻¹ MgSO₄·7 H₂O, 0.036 g L⁻¹ CaCl₂·2 H₂O, 0.006 g L⁻¹ citric acid, 0.04 g L⁻¹ K₂HPO₄, 0.006 g L⁻¹ iron ammonium citrate, 0.001 g L⁻¹ Na₂EDTA, 0.02 g L⁻¹ Na₂CO₃, 1 mL L⁻¹ BG11 trace elements (1000×), 0.3% Na₂S2O₃, 10 mM HEPES (pH 8), 1.5% Agar; BG11 trace elements (1000×): 2.86 g L⁻¹ boric acid, 1.8 g L⁻¹ MnCl₂·4 H₂O, 0.22 g L⁻¹ ZnSO₄·7 H₂O, 0.39 g L⁻¹ Na₂MoO₄·2 H₂O, 0.08 g L⁻¹ CuSO₄·5 H₂O, 0.05 g L⁻¹ Co(NO₃)₂·6 H₂O

FIG. 1 shows a bioreactive extraction of photosynthetically produced oxygen by the alkane monooxygenase enzyme system AlkBGT which was introduced genetically into the cyanobacterial strain Synechocystis sp. PCC6803. The photosynthetic light reaction was induced by illumination (luminous intensity of 50 μmol m⁻² s⁻¹), while the control reaction took place without illumination. Terminally hydroxylated methyl nonanoate was detected as oxygenated product. CDW=cell dry weight

For this, the alkane monooxygenase enzyme system AlkBGT was introduced into the cyanobacterial strain Synechocystis sp. PCC6803 (via the plasmid pRSF_Ptrc1O:BGTII, see production of the modified strain above), which is a potential biocatalyst for photosynthetic hydrogen production. After converting the reaction system from aerobic to anaerobic conditions and after adding methyl nonanoate as substrate, the formation of oxygenated product could be detected when illuminated (FIG. 1).

As no product formation was observed at reaction conditions in darkness, the results demonstrate that the oxygen needed for the reaction came from the photosynthetic light reaction. The specific activity was 0.9±0.1 μmol_{oxygenated substrate}min⁻¹ g_{CDW}⁻¹ for the first 30 minutes. The oxygen formation rate at the same luminous intensity of 50 μmol m⁻² s⁻¹ was 3.7±0.5 μmol_{O2} min⁻¹ g_{CDW}⁻¹ (without addition of substrate). Thus almost 25% of the produced oxygen could be absorbed enzymatically with the non-optimized biocatalyst. When observing the possible dilution of the oxygen from the aqueous phase into the gas phase (aqueous:gas phase ratio 1:10, dimensionless Henry volatility H_{CC}=caq/cgas for O₂ in water: 0.0297 at 25° C. (Sander 2015)) it is clear that the molecular oxygen has already been captured at the point of its production and bound into the substrate before escaping into the gas phase. An optimization of the oxygenation system (for example by increasing the enzyme content in the cell) is possible and would increase the proportion of extracted oxygen.

### Example 2

### Technical Application—Tube Bundle Reactor Concept

The technical implementation of the described invention takes place preferably using a concept in which an illuminated tube bundle reactor, which contains suspended or immobilized phototropic cells, is used. The tube bundle reactor is scaled up on an industrial scale by simply increasing the number of microcapillaries used. As a special form of immobilized cells, a biofilm-based design is chosen which proved achievable for the cyanobacteria species Synechocystis sp. PCC6803 (David, C., K. Buhler and A. Schmid (2015). “Stabilization of single species Synechocystis biofilms by culivation under segmented flow.” J Ind Microbiol Biotechnol 42(7): 1083-1089). This technical implementation offers a continuous production system which contains the photosynthetic water splitting, hydrogen production, oxygen extraction, as well as substrate oxidation.

Downstream preparation is then made possible by capturing the molecular hydrogen via the gas phase and, when using wastewater as substrate, by guiding the treated wastewater back to the continuing purification process. If, on the other hand, a combined biocatalytic production process is intended, a separation of the product with increased value creation is to be carried out for example by using an organic carrier phase. The following examples contain assumptions and values which are adapted according to the specific application and the chosen framework conditions, and show the potential of the invention.

Specific Activity in Respect of Water Splitting by the PSII

The microorganisms used have a content of approx. 1% of photosystem II, wherein photosystem II has a molar mass of 350 kDa (g_{CDW}⁻¹, molecular weight PSII (Shen, J. R. (2015). “The structure of photosystem II and the mechanism of water oxidation in photosynthesis.” Annu Rev Plant Biol 66: 23-48). The highest in vitro measured value is indicated by Dismukes et al. (Dismukes, G. C., R. Brimblecombe, G. A. Felton, R. S. Pryadun, J. E. Sheats, L. Spiccia and G. F. Swiegers (2009). “Development of bioinspired Mn₄O₄-cubane water oxidaton catalysts: lessons from photosynthesis.” Acc Chem Res 42(12): 1935-1943) at 1000 s⁻¹. This leads to a specific activity of the PSII of 1700 μmol_{H20splitting} per minute per g_{CDW}⁻¹. From this, the result in respect of hydrogen production is an oxygen consumption and a substrate oxidation by the organism of 850 μmol_{H2/O2/substrate} per minute per g_{CDW}⁻¹.

Product Yield by a Tube Bundle Reactor Process

10 g_{CDW} L⁻¹ biomass with a volumetric productivity of 0.51 mol_{H2/O2/substrate} L⁻¹ h⁻¹ was used on 20,000 microcapillaries each 2 m long and 5 mm in diameter. A total volume of 785 L (39.3 mL capillary⁻¹) was the result. With 2920 hours of daylight per year (light availability of 8 h per day, 365 days) the system produces 1169 kmol hydrogen, and oxidized substrate, per year.

Energy Yield

The condition applies that the process is not limited by hydrogenase. On the basis of a molecular weight of 2 g/mol, hydrogen has a volumetric product activity of 1.02 g_{H2} L⁻¹ h⁻¹. This results in a product yield of 2338 kg hydrogen per year, of which 60%, thus 1402 kg, can be recovered. With an underlying energy content of 33.3 kWh per kilogram hydrogen (Wikipedia), a total of 46,686 kWh per year of energy can be generated using the process according to the invention.

1. A method for the bioreactive extraction of produced oxygen from a reaction chamber, comprising the following steps:
irradiating at least one phototropic microorganism under anaerobic conditions in a reaction chamber, and
in situ bonding of produced oxygen by means of the microorganism by oxidation of an oxygen-utilizing substrate.

2. The method according to claim 1, wherein the at least one phototropic microorganism has or produces at least one oxygen-converting enzyme.

3. The method according to claim 2, wherein the at least one enzyme is selected from the group hydrogenase, nitrogenase and/or oxidoreductase.

4. The method according to claim 1, wherein, during irradiation of the at least one phototropic microorganism, hydrogen or methanol is also released in addition to oxygen.

5. The method according to claim 1, characterized in that the oxidoreductases are selected from the group of oxidases or oxygenases, preferably monooxygenases, dioxygenases or coenzyme-independent oxygenases.

6. The method according to claim 1, wherein algae or cyanobacteria, preferably cyanobacteria, are used as phototropic microorganism.

7. The method according to claim 1, characterized in that a, in particular genetically modified, cyanobacterial strain is used as microorganism, which stem has the alkane monooxygenase enzyme system AlkBGT.

8. The method according to claim 1, characterized in that the genetically modified cyanobacterial strain Synechocystis sp. PCC6803, which has or produces the alkane monooxygenase enzyme system AlkBGT, is used.

9. The method according to claim 8, characterized in that methyl nonanoate is used as substrate.

10. A use of phototropic microorganisms which, in addition to hydrogenases, nitrogenases or oxidoreductases, as hydrogen-producing enzyme, additionally have or produce oxidoreductases as oxygen-converting enzymes, for obtaining hydrogen (H₂) or methanol and oxygen (O₂) from water or aqueous liquids and solutions and for simultaneous in-situ removal of the oxygen (O₂) using corresponding substrates which form products with the oxygen.

11. A photobioreactor comprising the genetically modified cyanobacterial strain Synechocystis sp. PCC6803 which has or produces the alkane monooxygenase enzyme system AlkBGT.


SEQUENCE LISTING (13 sequences)

SEQ ID NO: 1 - DNA, 76 bp, Artificial Sequence
  Primer
tccggctcgtataatgtgtggaattgtgagcggataacaatttcacacatactagtaccaggcatcaaataaaacg

SEQ ID NO: 2 - DNA, 19 bp, Artificial Sequence
  Primer
tataaacgcagaaaggccc

SEQ ID NO: 3 - DNA, 63 bp, Artificial Sequence
  Primer
tgatttctggaattcgcggccgctttctagattgacaattaatcatccggctcgtataatgtg

SEQ ID NO: 4 - DNA, 50 bp, Artificial Sequence
  Primer
acaccttgcccgtttttttgccggactgcagtataaacgcagaaaggccc

SEQ ID NO: 5 - DNA, 18 bp, Artificial Sequence
  Primer
cttttcctcgtagagcac

SEQ ID NO: 6 - DNA, 68 bp, Artificial Sequence
  Primer
gagccacccgcagttcgaaaaatagtactagagtagtggaggttactagatggcaatcgttgttgttg

SEQ ID NO: 7 - DNA, 21 bp, Artificial Sequence
  Primer
atcaggtaattttatactccc

SEQ ID NO: 8 - DNA, 51 bp, Artificial Sequence
  Primer
ctatttttcgaactgcgggtggctccaagcgctcttttcctcgtagagcac

SEQ ID NO: 9 - DNA, 79 bp, Artificial Sequence
  Primer
ctttcgttttatttgatgcctggtactatttttcgaactgcgggtggctccaagcgctatcaggtaattttatactccc

SEQ ID NO: 10 - DNA, 50 bp, Artificial Sequence
  Primer
gggaggtattggaccgcattgaactctagtatataaacgcagaaaggccc

SEQ ID NO: 11 - DNA, 50 bp, Artificial Sequence
  Primer
acgagccggatgattaattgtcaatctagagccaggcatcaaataaaacg

SEQ ID NO: 12 - DNA, 19 bp, Artificial Sequence
  Primer
ccatcaaacaggattttcg

SEQ ID NO: 13 - DNA, 20 bp, Artificial Sequence
  Primer
tgccacctgacgtctaagaa